(19) United States
(12) Patent Application Publication
(10) Pub. No.: US 2023/0221341 A1
(43) Pub. Date: Jul. 13, 2023

(54) METHOD FOR EVALUATING THE METABOLIC ACTIVITY OF A NON-CANCER CELL

(71) Applicant: UNIVERSITA' DEGLI STUDI DI UDINE, Udine (IT)

(72) Inventors: Fabio DEL BEN, PORDENONE (IT); Matteo TURETTA, VENEZIA (IT)

(21) Appl. No.: 18/014,872

(22) PCT Filed: Jul. 2, 2021
(86) PCT No.: PCT/IT2021/050206

§ 371 Date: Jan. 6, 2023

(30) Foreign Application Priority Data
Jul. 7, 2020 (IT) 102020000016429

(51) Int. Cl. G01N 33/84; G01N 33/50; G01N 33/569

(52) U.S. Cl. CPC G01N 33/84; G01N 33/5038; G01N 33/56972

(57) ABSTRACT

Method for evaluating metabolic activity of non-tumor cells in a biological fluid sample via detection of extra-cellular acidification rate.

### FIELD OF THE INVENTION

Embodiments described herein relate to a method for evaluating metabolic activity of a non-tumor cell, in particular for clinical and diagnostic purposes.

### BACKGROUND OF THE INVENTION

The complete blood count and leukocyte differential count are the most frequently requested clinical laboratory tests worldwide (1,2). Leukocyte differential count provides clinically useful parameters in the context of diagnosis, monitoring and treatment of infectious, autoimmune, neoplastic and degenerative pathologies. Automatic hematology analyzers use single-cell measurements to create 2- or 3-dimensional scattergrams to count and differentiate the different subtypes of circulating leukocytes.

Most popular technologies are based on the measurements of nucleic acids (Sysmex), enzymatic activity (Siemens) or morphometric/physical parameters (Beckman).

Recent studies show that, besides the above-mentioned parameters, metabolism varies across leukocyte subpopulations (3). Furthermore, a change in metabolism is required to drive some effector function, particularly in lymphocytes, and is therefore an indicator of “functional” activity (4,5). Metabolism seems to be a rather unexplored aspect of leukocyte biology in the context of differential count and clinically oriented biomarkers.

However, current methods for measuring the metabolic activity of circulating leukocytes require the isolation and culture of the cells of interest, and are, therefore, relatively time- and resources-consuming procedures, possibly altering the “native-state” of circulating leukocytes.

Blood analysis is also extensively used in prenatal diagnosis as screening test for detecting chromosomal abnormalities that may affect the fetus by isolating fetal cell-free DNA (cfDNA) from the mother blood stream. The practice is considered non-invasive because it requires drawing blood only from the pregnant women and does not pose any risk to the fetus.

The fetal cfDNA isolated is used to detect, in particular, aneuploidy or others additional chromosomal disorders (e.g. deleted or copied section of chromosomes).

Despite the advantage of a non-invasive practice, cfDNA circulates in the blood in fragmented form, moreover, fetal cfDNA is only a slight proportion compared to the amount of total isolated cfDNA that contain also mother cfDNA in high amounts, making, therefore, fetal cfDNA difficult to analyze even with recent technologies.

For those reasons this screening test may cause false negative results that need to be confirmed by an invasive test increasing, therefore, the demand of new reliable non-invasive methods for prenatal screening and/or diagnosis.

Oxygen consumption and proton production rates (PPR) are measured as correlate with mitochondrial function and glycolysis, respectively, that in turn, may be correlated with cells' metabolic activity.

The direct or indirect measurement of the production of acidity raising molecules e.g. lactic acid, lactate ions and protons, that are correlated with extracellular pH, is referred as Extracellular Acidification Rate (ECAR). The higher the ECAR value, the higher is acidity raising molecules production and the lower is pH.

The limitation is that PPR is assessed by detecting the pH change of the extracellular medium of a cell culture well, thus measuring an average activity of cultured cells, without the possibility to describe cellular heterogeneity through single-cell analysis.

A method to detect Circulating Tumor Cell (CTC) in the blood stream evaluating the extracellular pH at single-cell level is described in EP-B-3.084.434. However, the detection of circulating tumor cells presents very different aspects and problems compared to the detection of non-tumor cells, in particular leukocyte cells or fetal cells. In particular, circulating tumor cells are considered rare cells and, therefore, an important difference is the quantity of circulating tumor cells subject to detection, which is minimal, even a few units, in particular in the order of from 1 to 10 cells/ml of blood, compared to non-tumor cells, where the cells analyzed are for example in the order of 10″4-10″6 cells/ml of blood.

Furthermore, document US-A-2019/0086391 describes an integrated method of cell culture and measurement of extracellular pH and oxygen of a plurality of cultured cells. This method, however, does not have sufficient sensitivity to go as far as measuring a single cell, even less can it be used to recognize and isolate cells with different metabolisms inside a population. Moreover, this known document does not apply to laboratory medicine and diagnostics on samples of body fluids.

U.S. Pat. No. 8,728,758 describes a method to monitor and analyze metabolic activity profiles of cells and corresponding diagnostic and therapeutic uses. The technique described measures a range of PBMC cells from cancer patients from 10\hat{ }3 to 10\hat{ }10 cells. In particular, the analysis is carried out on a single sample of cells, and not on single cells, on which sample multiple measurements can be made, using multiple wavelengths in sequence in spectrophotometry.

There is therefore a need to improve a method for evaluating metabolic activity of a non-tumor cell, which overcomes at least one of the drawbacks in the art.

The Applicant has devised, tested and embodied the present invention to overcome the shortcomings of the state of the art and to obtain these and other purposes and advantages.

### REFERENCES

1. Buttarello M, Plebani M. Automated blood cell counts: state of the art. Am J Clin Pathol. 2008 July; 130(1):104-16.
2. Horton S, Fleming K A, Kuti M, Looi L-M, Pai S A, Sayed S, et al. The Top 25 Laboratory Tests by Volume and Revenue in Five Different Countries. Am J Clin Pathol. 2019 Apr. 2; 151(5):446-51.
3. Kramer P A, Ravi S, Chacko B, Johnson M S, Darley-Usmar V M. A review of the mitochondrial and glycolytic metabolism in human platelets and leukocytes: Implications for their use as bioenergetic biomarkers. Redox Biol. 2014 Jan. 10; 2:206-10.
4. Dimeloe S, Burgener A, Grahlert J, Hess C. T-cell metabolism governing activation, proliferation and differentiation; a modular view. Immunology. 2017 January; 150(1):35-44.
5. Gubser P M, Bantug G R, Razik L, Fischer M, Dimeloe S, Hoenger G, et al. Rapid effector function of memory CD8+ T cells requires an immediate-early glycolytic switch. Nat Immunol. 2013 October; 14(10):1064-72.

### SUMMARY OF THE INVENTION

The present invention is set forth and characterized in the independent claims, while the dependent claims describe other characteristics of the invention or variants to the main inventive idea.

The present invention provides a method for evaluating metabolic activity of a non-tumor cell in a biological fluid sample, comprising:

encapsulating each single non-tumor cell in a volume of about 10 pL to 10 nL of said fluid,

incubating said volume at a temperature of from 4° C. to 37° C. for at least 1 minute,

detecting a pH and/or a concentration of at least one acid molecule, for example lactic acid, lactate ions and protons, within said incubated volume, which correlates with said extra-cellular acidification rate of said cell.

According to an aspect of the invention, a decrease in the pH and/or an increase in the concentration of the at least one acid molecule with respect to a reference pH and/or concentration determined for the same volume before incubating, indicates an increase, or a change in general, of the metabolic activity of said non-tumor cells present in said biological fluid sample.

According to an aspect of the invention, the reference pH and/or concentration is determined via measurement of the pH and/or concentration of an encapsulated volume of said fluid free of non-tumor cells.

According to an aspect of the present invention, the biological fluid sample is blood or its derivatives.

According to an aspect of the present invention, the non-tumor cells present in the biological fluid sample object of the method described here are from 10^{\hat{ }4} to 10^{\hat{ }6} cells/ml of sample.

According to an aspect of the present invention, said non-tumor cells are leukocyte cells and said evaluation of metabolic activity is used for the functional classification of the leukocyte cells.

According to an aspect of the present invention, said method comprises obtaining information on the cell type by means of at least one marker configured to allow a discrimination between different leukocyte populations.

According to an aspect of the present invention, the metabolic activity may reflect activation of some biological process or pathways activation, or even alteration, of a cell and, thus, its ability to carry out their function.

For example, leukocytes, as immunity response is required, e.g. in presence of infection or cancerous cells or, generally, inflammation, turn their state from a quite state to an activate state that is associated with an alteration of their metabolism.

Similarly, fetal cell display an altered metabolism with respect to adult cell. It is known that tumor cells have an extremely high ability to produce acid molecules compared to non-transformed or non-tumor cells, such that it is possible to use the measure of extracellular pH at single cell level to identified circulating tumor cells (CTCs) in the blood stream.

Inventors have surprisingly discovered that, despite lower compared to that of CTCs, extracellular acidification rate of non-tumor cells is measurable with the method of the present invention and may correlate to the healthy status of a subject and/or it may be used to isolate particular subpopulation of non-tumor cells for further analysis.

The method according the present disclosure may supply information in particular of glycolytic activity of a non-tumor cell.

According to an aspect of the invention, the detected pH and/or concentration are used for identification and/or classification of said encapsulated non-tumor cell.

The method may be used as diagnostic tool to assay a blood sample of a subject to obtain information from leukocytes contained within, wherein such information may be used in several clinical fields, for instance:
infective disease monitoring, or non-evaluable suspect of infective disease (e.g. sepsis, post-traumatic or post surgery fever);
autoimmune disease monitoring (non-evaluable chronic inflammatory disease);
degenerative pathologies monitoring or detection;
onco-hematologic pathologies (liquid or solid tumors).

The method may also be used to possibly also detect circulating fetal cells in a blood sample of a pregnant woman. Advantageously, the method may provide isolation of the fetal cells, obtaining an enriched sample of fetal cells that can be used to perform prenatal diagnostic test(s).

The enriched sample of fetal cells provides a pure source of fetal DNA, not so far obtainable in non-invasive manner, which can be used for prenatal/genetic analysis.

According to an aspect, the method comprises isolating cells from said biological fluid.

The present invention, since it is particularly focused on detecting non-tumor cells, in particular at least leukocyte cells, provides to detect and analyze a very high number of cells, for example in the order of 10^{\hat{ }4}-10^{\hat{ }6} cells/ml of blood, instead of circulating tumor cells which instead are rare, only a few units, in the order of 1-10 cells/ml of blood. In the context of this number of non-tumor cells, it is possible to carry out a profiling of the population of cells detected. For example, in the case of leukocytes, there are different leukocyte populations that exhibit different metabolisms and metabolic changes connected to their biology. A lymphocyte, for example, takes on characteristics similar to a neoplastic cell to activate, while a neutrophil has a high acidifying activity that it loses if damaged. The method of the present invention allows the functional classification of cellular subpopulations of clinical interest which can be correlated to a determinate disease or a suspected disease or a clinical decision for managing a patient.

The detection and analysis of the method according to the present invention is therefore aimed not only at identifying the different cells present, but also at profiling them based on metabolic aspect, exploiting the pH, and therefore allowing to discriminate between different leukocyte populations or to identify new unknown classes of leukocytes that share the same metabolic characteristic, which is not possible with the immunophenotypic approach alone.

In particular, this discrimination occurs through the possible aid of markers that help to discriminate the various leukocyte populations and that can use physical quantities such as optical measurements, optics, such as light scattering at different angles, electrical, colorimetric or other measurements, or antibody markers. The output of the method described here is therefore a series of thousands of “single cell” measurements with which the profiles of the different leukocyte populations can be built.

For example, if the marker is associated with a measurable physical quantity, it can be of the type that can be used coupled with a potentiometric, conductometric, capacitive, amperometric, voltametric or optical measurement, for example based on fluorescence, based on chemiluminescence or electrochemiluminescence.

One output of the method described here can therefore also be represented by a series of scatter plot graphs in which the relationships between pH and individual markers can be visualized, and in which an analyst will be able to recognize “typical” patterns or quantifications given by the segmentation of the graph that are associated with patient outcomes, in a manner similar to current practice in flow cytometry. Unlike non-tumor cells object of the present description, in the case of tumor cells typically few cells are present, for example from 1 to 10 cells, which are insufficient to create a repeatable pattern. Furthermore, another output of the method described here can be a relational database where each row is a cell and each column a characteristic of that cell selected from pH, marker 1, marker 2, . . . marker n. This database will have a number of rows in the order of 10^{\hat{ }4}-10^{\hat{ }6} and columns in the order of 1-10 or more. This database structure supplies an excellent substrate of statistical analysis and data science techniques, using artificial intelligence routines for machine learning, that is, based on machine learning.

With this advanced data analysis, it is possible to obtain patient outcome predictions on the basis of subtle patterns invisible to the analyst's eye, or relationships between variables too complex to be noticed by the analyst. In the case of circulating tumor cells, the typical output is 1-10 cells/ml of blood, a number with which the methods that use artificial intelligence routines, in particular machine learning, are not able to obtain valid performances, nor is it possible to carry out satisfactory statistical analyzes on the population.

Advantageously, moreover, with the method described here it is also possible to couple to each encapsulated cell a captured image of the same cell as it passes an optical detection threshold. This image can be used as another element of the relational database as above, each image being associated with a row of the database, corresponding to a cell. This image, for each cell, can feed the artificial intelligence models and routines, in particular using machine learning, for the advanced statistical analysis as above.

Moreover, according to other embodiments, the method described here offers the further possibility of studying how the identified metabolic profiles vary under the influence of specific drugs. This solution can be implemented by analyzing the sample in parallel runs using different drugs on each occasion and comparing the profiles, or injecting the drug with microfluidic technology directly into the droplets defined by the volume in which the single encapsulated cells are encapsulated and carrying out measurements in series.

Furthermore, since the method described here, in the case in which cell isolation is provided, can make a large number of non-tumor cells available, each individually encapsulated and isolated on the basis of a metabolic parameter, it offers the advantage of being able to study single cells with a defined metabolic characteristic or populations homogeneous from the metabolic point of view with molecular biology techniques, which would not be possible on the specific metabolic side (extracellular acidification capacity).

### BRIEF DESCRIPTION OF THE DRAWINGS

The accompanying drawings relate to embodiments of the disclosure and are described in the following:

FIGS. 1A-1C are two dimensions plots of encapsulated leukocytes showing the effect of time and glucose administration on ECAR. In particular,

FIG. 1A refers to ECAR detection after 30 minutes of incubation of leukocytes;

FIG. 1B refers to a comparison of ECAR detection after three different time of incubation of leukocytes treated with 5 mM of glucose; FIG. 1C refers to a comparison of ECAR detection after two different time of incubation of leukocytes treated with and without glucose;

FIGS. 2A and 2B are two dimensions plots of encapsulated leukocytes showing the effect of drug treatment on ECAR. In particular FIG. 2A refers to ECAR detection after 120 minutes of incubation of leukocytes with or without glycolysis affecting drugs; FIG. 2B refers to ECAR detection after 120 minutes of incubation of leukocytes with or without leukocyte's activity stimulation drugs.

FIG. 3 are a series of two dimensions plots of encapsulated leukocytes showing the effect of hematological or not-hematological conditions on ECAR in leukocytes together with a healthy control.

### DETAILED DESCRIPTION OF SOME EMBODIMENTS

Reference will now be made in detail to the various embodiments of the invention. Each example is provided by way of explanation of the invention and is not meant as a limitation of the invention. For example, features illustrated or described as part of one embodiment can be used on or in conjunction with other embodiments to yield yet a further embodiment. It is intended that the present invention includes such modifications and variations.

It shall also be clarified that the phraseology and terminology used here is for the purposes of description only, and cannot be considered as limitative.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

Embodiments described herein relate to a method for evaluating metabolic activity of non-tumor cells present in a biological fluid sample of between 10^{\hat{ }3} to 10^{\hat{ }5} cells, via detection of extra-cellular acidification rate (ECAR), comprising:

encapsulating each single non-tumor cell in a volume of about 10 pL to 10 nL of said fluid,

incubating said volume at a temperature of from 4° C. to 37° C. for at least 1 minute,

detecting a pH and/or a concentration of at least one acid molecule, within said incubated volume, which correlates with said extra-cellular acidification rate of said cell.

According to an aspect of the invention, a decrease in the pH and/or an increase in the concentration of the at least one acid molecule, with respect to a reference pH and/or concentration determined for the same volume before incubating, indicates an increase, or a change in general, of the metabolic activity of said non-tumor cells present in said biological fluid sample.

Within the context of the present disclosure, the term “cell” refers to the smallest structural and functional unit of an organism, which is typically microscopic and consists of cytoplasm and a nucleus enclosed in a membrane.

In the present description, the term “non-tumor cell” refers to a cell which does not undergo, or is not affected by, any tumor features or hallmarks, in particular which is not recognized as a tumor cell by known tumor identification techniques, such as identification techniques based on immunocytochemicals methods, morphological criteria, cell behavior or DNA/RNA-abnormalities.

Moreover, in the present description, the term “non-tumor cell” and “cell” are interchangeable and in any case always indicate “non-tumor cell”, unless otherwise specified.

Furthermore, in the present description, an encapsulated volume of said biological fluid sample might be simply referred to as a “droplet”. Thus, an encapsulated non-tumor cell might be referred to as a droplet containing one non-tumor cell. On the other hand, an encapsulated volume of said biological fluid sample, which is free of non-tumor cells, might be referred to as a droplet free of non-tumor cells.

A microfluidic device may be used to encapsulate said volume of the biological fluid sample.

A microfluidic device as described in EP-B-3.084.434, which is hereby incorporated by reference, may be generally used to encapsulate said volume of the biological fluid sample; therefore, such a microfluidic device might be used to encapsulate one non-tumor cell in said volume, i.e. to obtain droplets each containing one non-tumor cell, and further to obtain an encapsulated volume of said biological fluid sample which is free of non-tumor cells.

In embodiments, the above mentioned reference pH and/or concentration is determined via measurement of the pH and/or concentration of an encapsulated volume of said fluid free of non-tumor cells, i.e. a droplet free of non-tumor cells.

In embodiments, the above-mentioned volume is in the form of a droplet within a droplet-based microfluidic device.

In embodiments, such microfluidic device may be used to screen the individual droplets using fluorescence-based techniques, or electrical systems, for example, capacitance measurements, electrochemical sensors, potentiometric nano-sensors or through direct molecules detection, for example, mass spectrometry or enzymatic assays.

Droplets flowing in the microfluidic device may be sorted, stored, re-injected into others microfluidic devices, fused with other droplets and the cells can be cultured within droplets.

The droplet volume may be suitable to allow droplets to flow in a fluidic system of flow cytometer like-architectures, for example, a conventional diagnostic hematological apparatus as a hemocytometer or flow cytometer.

The flowing of droplets in a fluidic system of flow cytometer like-architectures might need the modification of flowing condition, e.g. switching from aqueous to oil sheet fluid or encapsulating the oil droplet in an aqueous droplet.

In embodiments, detecting said pH and/or concentration may be performed in a hemocytometer or flow cytometer.

In embodiments, the cell may be encapsulated in the microfluidic device and injected in one of the aforementioned apparatuses provided with an optical setup or equipment suitable to excite the pH indicator in the droplet and to read its emission signal for carrying out the detection of change in pH. Therefore, the method may be implemented in routine diagnostic since hemocytometers, or flow cytometers form the standard equipment of a clinical laboratory.

According to embodiments, each non-tumor cell is encapsulated in a droplet that can be part of an aqueous emulsion in a microfluidic device.

In one embodiment, the droplet is a water-in-oil emulsion, nevertheless a double emulsion may be employed. Fluorous oil, such as HFE 7500 or FC-77 or FC-40 from 3M™ may be preferred due to their ability to store dissolved oxygen. The emulsion may be formed on-chip or separately.

In one embodiment, the biological fluid might be a body fluid and might be selected form the group comprising blood, serum, lymph, pleural fluid, peritoneal fluid, cerebrospinal fluid, urine, saliva.

In the case of blood, the method according to the present disclosure may comprise an initial step for removing of red blood cells in order to accelerate the throughput.

The incubation step may be carried out at room temperature, or generally between 4° C. and 37° C. The incubation time may be from at least one minute to 48 h. The incubation step, that is time and temperature incubation, may vary with respect to the subpopulation to assay.

The pH values can be determined by a pH-indicator, fluorescence-based techniques, electrical systems or through direct molecules detection as stated above.

The pH-indicator can be either pH-sensitive dye or an indicator that changes its absorption/emission spectrum while the pH changes. Examples of these indicators are pHrodo™ Green (Life Technologies), which fluoresces green at acidic pH, SNARF®-5F 5-(and-6) Carboxylic acid (Life Technologies), with the ratio between 580 nm and 640 nm fluorescence increasing at acidic pH, and pH-sensitive inorganic salt which aggregates to form microcrystals.

The method according to the present disclosure may also comprise irradiating the encapsulated non-tumor cell with light laser, said detected pH being function of an emitted signal of said irradiated encapsulated non-tumor cell.

According to the present disclosure, pH evaluation may be also carried out measuring the concentration of lactic acid or lactate ions or protons by using any technique known to the skilled person for such a purpose.

According to aspects of the present disclosure, the detected pH and/or measured concentration are used for identification and/or classification of said encapsulated non-tumor cell.

Therefore, the method according to the present disclosure allows the detection, at the level of each single encapsulated cell, of a cell in a particular functional state that it is known to be correlated to an altered ECAR activity. Since ECAR activity may mostly caused by glycolytic pathway activation and its degree of activation, the present method allows the study of glucose metabolism with respect to glycolytic activity.

In embodiments, the method according to the present disclosure may comprise a treatment step in which cells are treated with a glycolytic pathway affecting drug allowing emerging of other side biological process responsible for altering pH and/or concentration that may have clinical interest.

In possible embodiments, the method can also comprise, in particular, analyzing the variation of the metabolic profiles identified under the influence of specific drugs. In particular, evaluating the variation of metabolic profiles identified under the influence of specific drugs provides to carry out analysis of the sample in parallel runs using different drugs on each occasion and comparing the profiles, or injecting the drug with microfluidic technology directly into the droplets defined by the volume in which the single encapsulated cells are encapsulated and carrying out measurements in series.

Advantageously, the method according to the present disclosure may be applied in diagnostic routine to analyze biological fluids, in particular blood for instance, to detect particular cell subpopulation of clinical interest that may be correlated to a certain disease or a suspect of disease or a clinical decision for patient management.

In embodiments, the identification and/or the classification may be carried out on the basis of at least one pH reference threshold or range corresponding to an experimentally measured normal extracellular acidification rate of a particular cell population or subpopulation taken as reference.

In embodiments, the method may comprise an isolation step for sorting out, from the biological fluid sample, either the volume comprising the non-tumor cell or directly the non-tumor cell.

The isolation step is extremely useful to obtain a uniform cell population, with at least the same ECAR activity, on which is possible to perform further analysis. Furthermore, thanks to the isolation, a number of non-tumor cells is obtained, each one individually encapsulated and isolated on the basis of a metabolic parameter, which can be studied with molecular biology techniques, in particular studying single cells with a defined metabolic characteristic or populations that are homogeneous from a metabolic point of view.

In embodiments, the method according to the present disclosure may comprises obtaining information on the cell type by means of at least one marker configured to allow to discriminate between different leukocyte populations.

According to possible embodiments, obtaining information on the type of cell comprises contacting the biological fluid sample with one or more probes that act as an antibody marker, suitable to bond with an antigen expressed by the non-tumor cell in order to obtain cell type information.

According to embodiments, said one or more probes may be, or include, a known hematological CD marker in order to obtain immunophenotype information. The marker may be selected from a group comprising: CD3 (T-lymphocytes), CD4 (Th-lymphocytes), CD8 (Tc-lymphocytes), CD14 (monocytes), CD15 (granulocytes), CD19 (B-lymphocytes), CD45 (leukocytes) or other known markers. The probe may be associated to a fluorescent molecule selected from Alexa-Fluor dye, green fluorescent protein (GFP), fluorescein derivate such as fluorescein thiocyanate (FITC), tetrametil-rhodamine (TRITC), allophycocyanin (APC), or suchlike.

Alternatively, obtaining information on the cell type comprises using, as a marker, a detected physical quantity, in particular an optical quantity, such as light scattering at different angles, an electrical or colorimetric quantity.

According to the present disclosure, non-tumor cells are leukocytes cells and said evaluation of metabolic activity is used for the functional classification of leukocytes cells.

According to the present disclosure, the method allows identifying leukocyte with altered metabolism, i.e. activated or anergic leukocytes or other not defined leukocyte populations of clinical interest with respect to their ECAR.

With respect to quiescent leukocytes, normally found in physiological conditions, anergic leukocytes are functionally inactivated and unable to initiate a productive response even when antigen is encountered in the presence of full co-stimulation.

On the contrary, activated leukocytes are capable of triggering a respiratory burst and degranulation.

In embodiments, the one or more probes may be used to discriminate different leukocytes subgroups (neutrophils, lymphocytes, monocytes, or others) providing a functional classification of a particular subpopulation or to exclude them from analysis.

In further embodiments, other markers may be used to stain cell of non-hematological origin.

One embodiment of the method may provide a step of leukocytes grouping in different ECAR activity group. ECAR activity groups may be defined by one or more thresholds below or over a reference value or a reference interval measured experimentally on leukocytes isolated from healthy subjects.

Thresholds may vary between different leukocytes subpopulation.

In embodiments, leukocytes identified as having altered metabolism may be isolated for further analysis or in vitro culture.

In embodiments, the method comprises building a relational database where each row is a cell identified by means of the functional classification of leukocyte cells as above, and each column a characteristic of the cell chosen from pH and one or more of the markers, and subjecting said database to statistical analysis using artificial intelligence routines, in particular machine learning, in order to obtain patient outcome predictions on the basis of identified patterns or complex relationships between elements of said database. Examples of artificial intelligence routines for automatic self-learning that can be used are unsupervised learning techniques, or supervised learning techniques, such as artificial neural networks or support vector machines (SVMs), possibly combined with rule-based experts systems and/or with data-mining techniques.

With the method of the present invention it is also possible to couple to each encapsulated cell a captured image of the same cell as it passes an optical detection threshold. This image can be used as another element in the relational database described above, each image being associated with a row of the database, corresponding to a cell. This image, for each cell, can feed artificial intelligence models and routines, in particular using machine learning, in order to perform the advanced statistical analyses as above.

According to the present disclosure, non-tumor cells can possibly also be fetal cells and the evaluation of metabolic activity as above may be used for the identification of a fetal cell for using in prenatal screening or diagnosis.

In embodiments, the method according to the present disclosure may be used to evaluate metabolic activity for the identification of a fetal cell providing an enriched source of fetal cells for using in prenatal screening or diagnosis.

Briefly, the microfluidic device comprise means for encapsulating a cell in a droplet with a volume of about 10 pL to 10 nL of biological fluid and means for detecting pH and/or a concentration of at least one acid molecule selected, for example, from lactic acid, lactate ions and protons.

### EXPERIMENTAL EXAMPLES

Device Fabrication

The device was made of PDMS (polydimethylsilicone) bonded to a glass surface and silanized to make it hydrophobic, as reported in EP-B-3.084.434, which is hereby incorporated by reference. Standard lithography procedures were used in microfabrication.

Optical Setup

The optical setup for measuring droplet fluorescence consisted of an inverted microscope (Nikon). A 405 nm laser beam ran through a cylindrical lens to form a line crossing orthogonally the microfluidic channel, where droplets were excited, and fluorescence signal emitted was captured by a 40× objective (Olympus LUCPlanFLN, 40×/0.60), split with dichroic filter and detected through bandpass filters (579/34; 630/38 and 450/65) by Photo Multiplier Tubes (PMTs) (H957-15, Hamamatsu). Signal was amplified 1V/uA gain and detected by the acquisition system (National Instruments cRIO-9024, analog input module NI9223) with a 10 μsec scan rate.

Droplet generation and encapsulation of the cells Monodispersed droplets were generated in chips with 20 μm wide T-junction.

Continuous phase: 2% (w/w) surfactant (Krytox-Jeffamine-Krytox A-B-A triblock copolymer) in HFE-7500 (3M).

Dispersed phase: cell suspension (1-2 millions cells/mL) in Joklik's modified EMEM containing 15% Optiprep and 4 μM SNARF-5F. Flow rates were set at 600 μL/h for continuous phase and 300 μL/h for dispersed phase.

pH-Assay for Extracellular Acidification Rate Measurements

The pH-sensitive fluorescent dye SNARF-5F (Invitrogen) was used to measure the pH of each droplet. SNARF-5F respond to pH variation undergoing a wavelength shift in the emission spectra. For each droplet the ratio of emitted fluorescence intensities at 580 and 630 nm (580/630 ratio) of SNARF-5F is calculated. As the pH is more acidic, SNARF-5F fluorescence increases at 580 nm while decreases at 630 nm. pH of the droplet is indicated by 580/630 ratio.

Samples

Leftover samples selected from the daily routine were collected in K3-EDTA tubes (Kima, Padova). White blood cells (WBCs) were analyzed after lysing whole blood with lysis solution (BD Bioscences), according to manufacturer's protocol, centrifuged at 300 g×5 min and resuspended in working solution (Joklik's modified EMEM, optiprep 15% and 4 uM SNARF-5F) to obtain a concentration of 1-2 millions cells/mL. Pathological samples were selected according to hemocytometric results (Beckman Coulter D×H 900) and patient history.

Results

Results are displayed in figures from 1 to 3.

In particular, higher 580/630 ratio, the lower pH as indicated in table 1.

TABLE 1

| 580/630 | pH |
|---|---|
| ratio | value |
| 0.76 | 8 |
| 1.05 | 7.4 |
| 1.44 | 7.0 |
| 2.03 | 6.5 |
| 3.21 | 6 |
| 3.72 | 5.5 |
| 3.85 | 5 |

Basal Condition

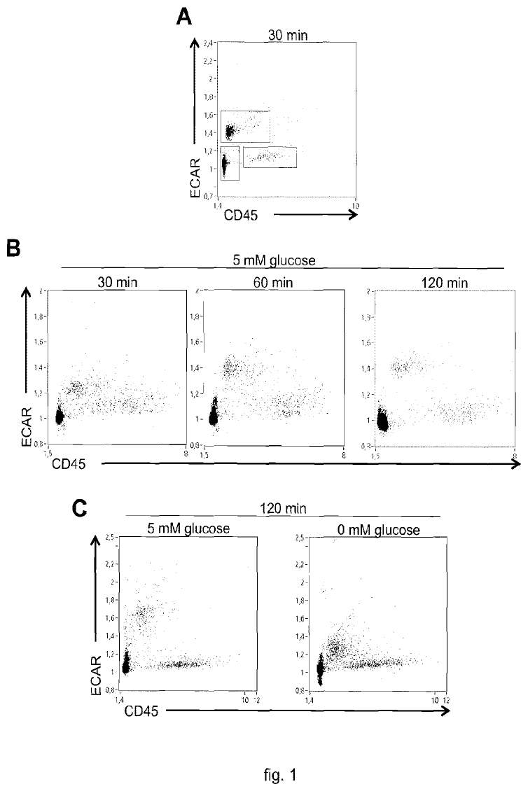

To study circulating leukocytes, derived from the peripheral blood of healthy donors, in their basal native condition, they were labeled with an anti-CD45 antibody and analyzed them with droplet microfluidics device. FIG. 1A shows representative plots obtained after incubating the leukocytes at 37° C. for 30 minutes.

Droplets consistently distributed on the plots in four clusters, clockwise:
a most abundant group with no CD45 signal and no ECAR activity that corresponds to empty droplets.
a major group having a CD45 low/ECAR high phenotype
a smaller group with a CD45 mid/ECAR very high phenotype
a major group with a CD45 high/ECAR low phenotype

As shown in FIG. 1A the neutrophils constitute the major subpopulation with a CD45 low/ECAR high phenotype, while monocytes that normally represent only a minor fraction, can be identified by a slightly increased CD45 expression and acidification potential.

Lymphocytes, on the other hand, are confirmed to be the population with the highest CD45 expression and lowest ECAR.

Of note, similar plots were obtained also without lysing the red blood cells, indicating that analysis of whole blood without any further manipulation is also possible.

Modulation of ECAR by Direct Control of Glycolysis

To ascertain that the observed effect could be attributed to glucose-dependent extracellular acidification and that the method was able to measure perturbations of such phenomenon, cells were observed over time and exposed to different conditions which are known to affect the glycolytic cascade.

Referring now to FIG. 1B, observing cells over time, up to 120 minutes of incubation, the difference in ECAR values between the clusters increased, as the ECAR activity showed a more significant time-dependent increase for the cells showing lower CD45 levels.

By comparing cells incubated in medium supplemented or not supplemented with glucose, as shown in FIG. 1C, leukocytes showed a strongly reduced ECAR activity in the absence of glucose. Accordingly, we could also observe a significative ECAR activity in the same cell population when the cells were incubated in PBS.

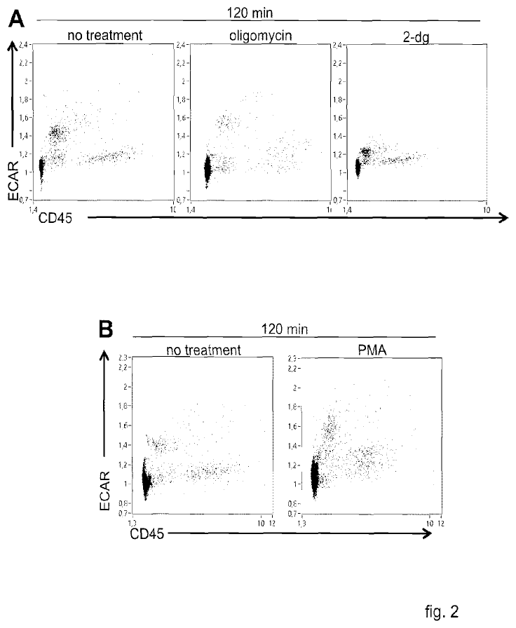

Referring to FIG. 2A, the cells were incubated in medium with or without the addition of oligomycin, which stimulates glycolysis by inhibiting mitochondrial ATP production, or with the glycolysis competitive inhibitor 2-deoxyglucose (2-DG), which suppresses glycolysis. Inventors found that oligomycin led to increased ECAR values of all cell populations, while 2-DG was, instead, able to significantly reduce ECAR, to a similar degree to what we could observe in the absence of glucose.

Modulation of ECAR by Immunostimulation of Cells Finally, as shown in FIG. 2B, the cells were also treated with PMA (Phorbol Myristate Acetate), a well known activator of protein kinase C (PKC), to stimulate leukocyte activity and oxidative burst initiation, and found that PMA was able to increase ECAR.

Pilot Exploration of Clinical Role of Single-Cell ECAR Analysis

The metabolic profile of circulating leukocytes has the potential to be a clinically relevant biomarker for the study of human disorders.

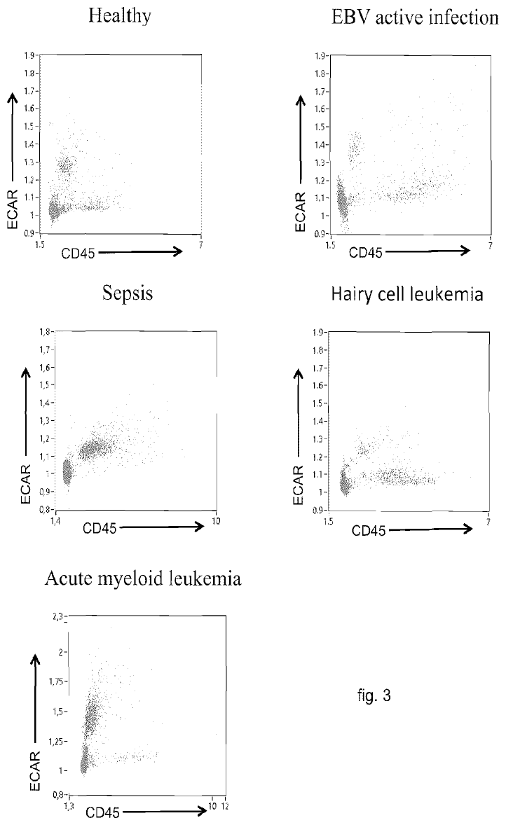

Referring to FIG. 3, initial screenings of the extracellular acidification activity of leukocytes in the context of hematological or not-hematological, pathological and paraphysiological conditions were performed. FIG. 3 shows a normal healthy control plot together with plots from patients with different lymphocyte abnormalities. By interpreting the plots to the light of data described above:
EBV infection plot shows a larger presence of lymphocytes with high ECAR,
Sepsis plot shows a gross prevalence of neutrophils, but the cluster has an altered shape (decreased variance of ECAR and increased variance of CD45). Lymphocyte cluster has also an altered shape.
Hairy cell leukemia plot shows a “double” lymphocyte population, one CD45low with a relatively higher ECAR, the other CD45high with a relatively lower ECAR.
Acute leukemia plot shows a neutrophil population with an altered shape, with a trend to higher ECAR.

It is clear that modifications and/or additions of steps may be made to the method as described heretofore, without departing from the field and scope of the present invention.

1. Method for evaluating metabolic activity of non-tumor cells present in a biological fluid sample, in particular blood or its derivatives, of 10^{\hat{ }4}-10^{\hat{ }5} cells/ml of sample, via detection of extra-cellular acidification rate, said method comprising:
encapsulating each single non-tumor cell in a volume of about 10 pL to 10 nL of said fluid,
incubating said volume at a temperature of from 4° C. to 37° C. for at least 1 minute,
detecting a pH and/or a concentration of at least one acid molecule, within said incubated volume, which correlates with said extra-cellular acidification rate of said cell, wherein a decrease in said pH and/or an increase in the concentration of said at least one acid molecule, with respect to a reference pH and/or concentration determined for the same volume before incubating, indicates a change of the metabolic activity of said non-tumor cells present in said biological fluid sample,
wherein said non-tumor cells are leukocyte cells and said evaluation of metabolic activity is used for the functional classification of the leukocyte cells;
wherein, moreover, said method comprises obtaining information on the cell type by means of at least one marker configured to allow a discrimination between different leukocyte populations.

2. Method as in claim 1, wherein said reference pH and/or concentration is determined via measurement of the pH and/or concentration of an encapsulated volume of said fluid free of non-tumor cells.

3. Method as in claim 1, wherein said detected pH and/or concentration are used for identification and/or classification of said encapsulated non-tumor cell.

4. Method as in claim 3, wherein said identification and/or said classification is carried out on the basis of at least one pH and/or concentration threshold or range corresponding to an experimentally measured normal extracellular acidification rate of a particular cell population or subpopulation taken as reference.

5. Method as in claim 1, wherein said method comprises an isolation step for sorting out, from said biological fluid sample, said volume comprising the non-tumor cell.

6. Method as in claim 1, wherein obtaining information on the cell type comprises contacting the biological fluid sample with one or more probes that act as an antibody marker, suitable to bond with an antigen expressed by the non-tumor cell in order to obtain cell type information.

7. Method as in claim 1, wherein obtaining information on the cell type comprises using, as a marker, a physical quantity detected, in particular an optical quantity, such as light scattering at different angles, an electric or colorimetric quantity.

8. Method as in claim 1, wherein said pH is detect by using a pH-indicator, in particular a pH-sensitive dye or an indicator that changes its absorption/emission spectrum while the pH changes.

9. Method as in claim 8, wherein said method comprises irradiating the encapsulated non-tumor cell with light laser, said detected pH being function of an emitted signal of said irradiated encapsulated non-tumor cell.

10. Method as in claim 1, wherein detecting said pH and/or concentration is performed in a hemocytometer or flow cytometer-like architectures.

11. Method as in claim 1, wherein said at least one acid molecule is selected from lactic acid, lactate ions and protons.

12. Method as in claim 1, said method comprising building a relational database in which each row is a cell identified by means of said functional classification of leukocyte cells and each column is a characteristic of said cell chosen from pH and one or more of said markers, and subjecting said database to statistical analysis using artificial intelligence routines, in particular machine learning, in order to obtain patient outcome predictions on the basis of identified patterns or complex relationships between elements of said database.

13. Method as in claim 12, said method comprising capturing an image of each encapsulated cell as it passes an optical detection threshold, said image being used as an additional element in said relational database, each image being associated with a row of the database in order to be subjected to said statistical analysis by means of artificial intelligence routines.

14. Method as in claim 1, said method comprising analyzing the variation of the metabolic profiles identified under the influence of specific drugs.

15. Method as in claim 14, wherein the evaluation of the variation of metabolic profiles identified under the influence of specific drugs provides to carry out an analysis of the sample in parallel runs using different drugs on each occasion and comparing the profiles, or injecting with microfluidic technology the drug directly into the droplets defined by the volume in which the single encapsulated cells are encapsulated and carrying out measurements in series.

16. Method as in claim 1, wherein said non-tumor cells are possibly also fetal cells and said evaluation of metabolic activity is used for the identification of a fetal cell for using in prenatal screening or diagnosis.

17. Method as in claim 2, wherein said detected pH and/or concentration are used for identification and/or classification of said encapsulated non-tumor cell.

18. Method as in claim 17, wherein said identification and/or said classification is carried out on the basis of at least one pH and/or concentration threshold or range corresponding to an experimentally measured normal extracellular acidification rate of a particular cell population or subpopulation taken as reference.
